(19) United States
(12) Patent Application Publication
(10) Pub. No.: US 2024/0081789 A1
(43) Pub. Date: Mar. 14, 2024

(54) SYSTEMS AND METHODS FOR SAMPLING A FLUID

(71) Applicant: Medtronic, Inc., Minneapolis, MN (US)

(72) Inventors: Cindy S. Roegge, Rogers, MN (US); Linda M. Page, Edina, MN (US)

(21) Appl. No.: 18/229,848

(22) Filed: Aug. 3, 2023

Related U.S. Application Data
(60) Provisional application No. 63/405,806, filed on Sep. 12, 2022.

(51) Int. Cl. A61B 10/00; A61M 39/02

(52) U.S. Cl. CPC A61B 10/0045; A61M 39/0208; A61B 2010/0077; A61M 2039/0202; A61M 2039/0235

(57) ABSTRACT

Systems and methods for sampling a fluid is provided. The system may include a port implanted in a patient and a catheter implanted in the patient and in fluid communication with the port. The catheter may enable fluid sampling from the patient and delivery of the fluid sample via the port.

### CROSS-REFERENCE TO RELATED APPLICATIONS

This application claims the benefit of U.S. Provisional Application No. 63/405,806, filed on Sep. 12, 2022, which application is incorporated herein by reference in its entirety.

### BACKGROUND

The present disclosure is generally directed to fluid sampling, and relates more particularly to fluid sampling using a fluid sampling system having an implanted catheter, a pump, and a port.

Fluid may be sampled from a patient for various reasons. In some cases, fluid is sampled repeatedly to track a treatment progress. Such fluid may be sampled using lumbar punctures, which may expose a patient to discomfort and risks associated with repeated lumbar punctures. Such lumbar puncture(s) may also expose a patient to radiation.

### BRIEF SUMMARY

Example aspects of the present disclosure include:

A system for sampling fluid according to at least one embodiment of the present disclosure comprises a port implanted in a patient; and a catheter implanted in the patient and in fluid communication with the port, the catheter enabling fluid sampling from the patient and delivery of the fluid sample via the port.

Any of the aspects herein, wherein the fluid is sampled from a patient having glioma.

Any of the aspects herein, wherein the fluid sampled is cerebrospinal fluid.

Any of the aspects herein, wherein the catheter is implanted to sample fluid from at least one of a spinal region or a brain region of the patient.

Any of the aspects herein, wherein the implanted port is implanted subcutaneously.

Any of the aspects herein, wherein the port comprises a septum in fluid communication with the catheter, and the system further comprises a needle configured to pierce the septum to collect the fluid sample.

Any of the aspects herein, further comprising a pump configured to pump the fluid sample to the port via the catheter.

Any of the aspects herein, further comprising a pump configured to pump the fluid sample to the port via the catheter.

A system for sampling cerebrospinal fluid according to at least one embodiment of the present disclosure comprises a port implanted in a patient; a catheter implanted in the patient and in fluid communication with the port, the catheter enabling sampling cerebrospinal fluid from the patient and delivery of the cerebrospinal fluid via the port; and a pump configured to pump the cerebrospinal fluid to the port via the catheter.

Any of the aspects herein, wherein the pump is implanted in the patient.

Any of the aspects herein, wherein the catheter is implanted to sample cerebrospinal fluid from at least one of a spinal region or a brain region of the patient.

Any of the aspects herein, further comprising a needle configured to selectively couple to the port to collect the fluid sample.

Any of the aspects herein, wherein the implanted port is implanted subcutaneously.

Any of the aspects herein, wherein the cerebral spinal fluid is sampled from a patient with glioma.

A method for sampling cerebrospinal fluid according to at least one embodiment of the present disclosure comprises implanting a catheter in a patient, the catheter in fluid communication with the cerebral spinal fluid to be sampled; implanting a port subcutaneously, the port in fluid communication with the catheter; and obtaining a sample of the cerebral spinal fluid from the port.

Any of the aspects herein, wherein obtaining the sample from the port comprises causing a pump to pump the cerebral spinal fluid from the spinal region to the port via the catheter.

Any of the aspects herein, wherein the pump is implanted in the patient.

Any of the aspects herein, wherein the sample of the cerebral spinal fluid is obtained from the port using a needle.

Any of the aspects herein, wherein the catheter is implanted in at least one of a spinal region or a brain region of the patient.

Any of the aspects herein, wherein the cerebral spinal fluid is sampled from a patient with glioma.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system implanted in a patient according to at least one embodiment of the present disclosure;

FIG. 2 is a block diagram of a system implanted in a patient according to at least one embodiment of the present disclosure;

FIG. 3 is a block diagram of a system according to at least one embodiment of the present disclosure;

FIG. 4 is a block diagram of a system according to at least one embodiment of the present disclosure;

FIG. 5 is a block diagram of a system according to at least one embodiment of the present disclosure; and

FIG. 6 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions (e.g., automatic retrieval of samples, automatically operating a pump, etc.) may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Cerebrospinal fluid (CSF) may be collected for a variety of reason and may provide information about a patient's treatment, disease progress, etc. For example, pediatric patients with glioma may need repeated spinal fluid sampling to track treatment progress. CSF may also be collected for evaluation of chemistries and/or biomarkers that may be used to evaluate disease progression, diagnose diseases and/or modify a patient's therapeutics. In another example, diseases such as Amyotrophic Lateral Sclerosis (e.g. ALS) or central nervous system diseases can be diagnosed from CSF and treatment of ALS can be tracked through repeated fluid sampling of the CSF. Thus, such repeated sampling and/or general ease of sampling allows for tracking a patient's response to treatment for patients with, for example, glioma or ALS, diagnosing potential diseases, and tracking disease progression. Conventional methods for collecting CSF in patients include repeat lumbar punctures, which come with risk, discomfort, and burdens medical resource. Further, lumbar punctures may use X-ray imaging to complete the process, thus exposing patients to radiation. Thus, according to at least one embodiment of the present disclosure, an implanted port system to sample the CSF could eliminate or significantly reduce the exposure to anesthesia and radiation needed to be used during a lumbar puncture. The implanted port system for CSF collection in patients with glioma, ALS, or any other disease can be created using an intrathecal port with a catheter and a refill kit, also referred to as a port administration kit. The intrathecal port can be implanted subcutaneously and attached to an intrathecal catheter. The refill kit or port administration kit can be used to access the implanted port system through the patient's skin.

Embodiments of the present disclosure provide technical solutions to one or more of the problems of (1) obtaining multiple, repeated fluid sample(s) from a patient, (2) reducing radiation and anesthesia exposure and medical risk to a patient, (3) increasing a patient's comfort during multiple, repeated fluid sampling, (4) enabling tracking of a patient's treatment progress (e.g., for patients with glioma, amyotrophic lateral sclerosis, or any diagnosis), and (5) reducing burden on medical resources.

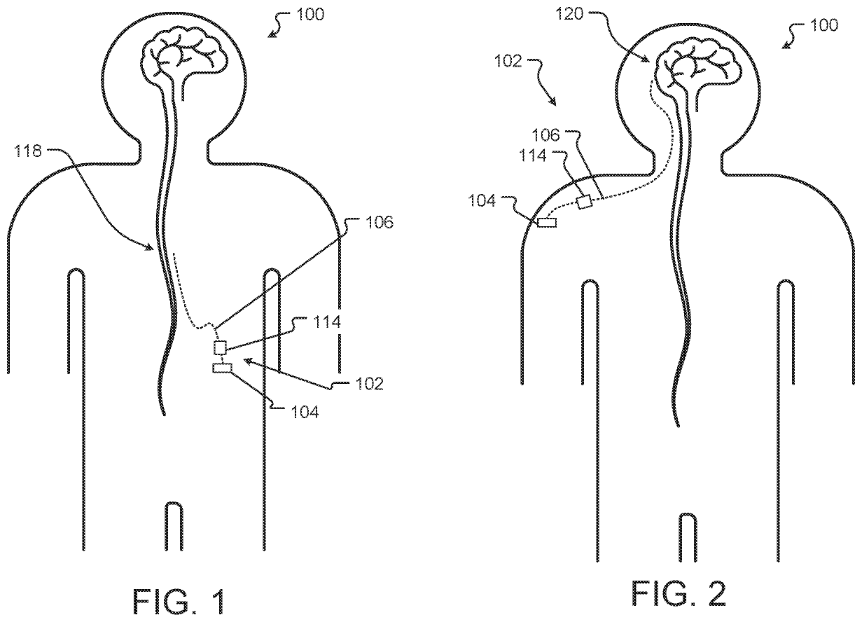

Turning first to FIG. 1, a block diagram of a patient 100 and a fluid sampling system 102 according to at least one embodiment of the present disclosure is illustrated. It will be appreciated that the fluid sampling system 102 enables both fluid sampling from the patient 100 and/or delivery of therapeutics to the patient 100. In particular, the fluid sampling system 102 enables repeated fluid samples of CSF from the patient 100 and in some instances, avoids performing an intrusive lumbar puncture. It will be appreciated that in other embodiments, the fluid sampling system 102 can collect fluid sample from, for example, an arterial space or vascular space. As previously described, the CSF may be useful in tracking treatment progress in glioma patients. In other instances, obtaining CSF—whether repeatedly or in a single instance—may be useful for evaluating the CSF chemistry and potential biomarkers for disease progression, disease diagnostics, and therapy modification. For example, diseases such as Amyotrophic Lateral Sclerosis (e.g. ALS) or central nervous system diseases can be diagnosed from CSF and treatment of ALS can be tracked through repeated fluid sampling of the CSF. Thus, such repeated sampling and/or general ease of sampling allows for tracking a patient's 100 response to treatment for patients with, for example, glioma or ALS, diagnosing potential diseases, and tracking disease progression.

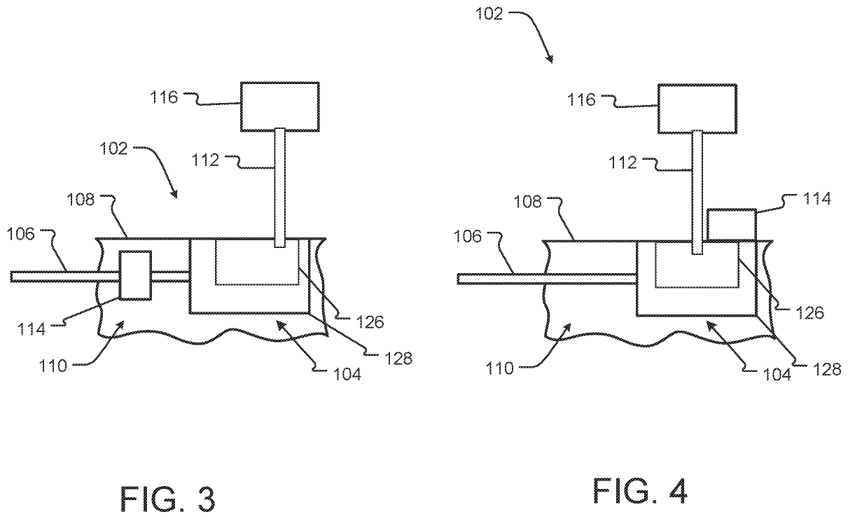

The fluid sampling system 102 may comprise a catheter 106, a pump 114, and a port 104 having a septum 126. It will be appreciated that in some embodiments, the fluid sampling system 102 may not include the pump 114. The catheter 106 may enable fluid sampling from the patient 100 and delivery of the fluid sample via the port 104 and the pump 114 may be configured to pump the fluid sample to the port 104 via the catheter 106. The catheter 106 may also enable delivery of therapeutics to the patient 100 and the pump. The fluid sampling system 102 may include additional components such as, for example, a needle 112 (shown in FIGS. 3 and 4), a syringe, a reservoir 116 (shown in FIGS. 3 and 4), or any other component.

Some or all components of the fluid sampling system 102 may be implanted in the patient 100. In the illustrated embodiment, the catheter 106, the pump 114, and the port 104 are implanted in the patient 100, though in other embodiments in which the fluid sampling system 102 includes the pump 114, the pump 114 may not be implanted in the patient 100 (as described in FIG. 4). In the illustrated embodiment, the catheter 106 may be implanted in a spinal region 118 of the patient 100. More specifically, the catheter 106 may be implanted intrathecally to enable sampling of the CSF. In other embodiments, the catheter 106 may be implanted anywhere in the patient 100 and enable sampling of any fluid from the patient 100. In the illustrated embodiment the pump 114 and the port 104 are implanted near an abdomen of the patient 100, though it will be appreciated that the pump 114 and the port 104 may be implanted anywhere in the patient 100.

Turning to FIG. 2, a block diagram of the patient 100 and the fluid sampling system 102 according to at least one embodiment of the present disclosure is illustrated. FIG. 2 is substantially similar to FIG. 1 except for placement of the fluid sampling system 102. In the illustrated embodiment, the catheter 106 is implanted near a brain region 120 of the patient 100 to enable sampling of the CSF. In such embodiments, the pump 114 and/or the port 104 may be positioned along an arm region of the patient 100. It will be appreciated that in other embodiments, the catheter 106, the pump 114, and/or the port 104 may be implanted or positioned anywhere on the patient 100.

Turning to FIGS. 3 and 4, a block diagram of the fluid sampling system 102 in a first configuration and a second configuration are shown respectively. As described above, the fluid sampling system 102 comprises the catheter 106, the pump 114, and the port 104. The catheter 106 may be or comprise, for example, the Medtronic Ascenda™ Catheter. In such embodiments, the port 104 may be designed to connect with and be in fluid communication with the Ascenda™ Catheter. In other embodiments, the catheter 106 may be any catheter 106 that is implantable in a patient 100 and enables fluid sampling from the patient 100 and/or delivery of therapeutics to the patient 100 or any intrathecal catheter implantable in the patient 100 that enables fluid sampling of, for example, CSF and/or delivery of therapeutics to the patient 100. As previously described, the catheter 106 may be implanted anywhere in a patient 100 including, for example, intrathecally in the spinal region 118 of the patient 100, in the brain region 120 of the patient 100, etc.

The fluid sampling system 102 also includes the pump 114. In some embodiments, the pump 114 may be or comprise the Medtronic SynchroMed™ Pump. In other embodiments, the pump 114 may be or comprise any pump 114 configured to pump sampling fluid from the patient 100 or therapeutics to the patient 100. As previously described, the pump 114 may be implanted in the patient 100, as shown in FIG. 3, or positioned outside of the patient 100, as shown in FIG. 4. In some embodiments, the fluid sampling system 102 may not include the pump 114.

The fluid sampling system 102 also includes the port 104. The port 104 comprises a housing 128 and a septum 126 disposed in the housing 128. The septum 126 may be in fluid communication with the catheter 106. In the illustrated embodiment, the port 104 is implanted subcutaneously 110 under a surface 108 of the patient's 100 skin. In other embodiments, the port 104 may be implanted anywhere on the patient 100 and may be implanted at the surface 108 or above the surface 108 of the patient's 100 skin. The port 104 is in fluid communication with the catheter 106 and provides for easy and repeatable access to fluid samples from the patient 100. Similarly, the port 104 provides for easy and repeatable access to the patient 100 to deliver therapeutics. The port 104 may be in fluid communication with the catheter 106 via one or more connectors. In some embodiments, the port 104 can be coupled to the catheter 106 via the one or more connectors and without sutures. The port 104 may also comprise a port as described in patent application Ser. No. 17/085,682; application Ser. No. 17/085,562; application Ser. No. 17/556,571; and/or application Ser. No. 16/949,418, which are incorporated by reference in their entireties.

The fluid sampling system 102 may also include a needle 112 configured to access the port 104. In embodiments where the port 104 is implanted below the surface 108 of the patient's 100 skin, the needle 112 is configured to pierce the patient's skin and the septum 126 until the needle 112 contacts a needle stop in the port beneath the septum 126. Once the needle 112 is has reached the stop, the needle 112 is in fluid communication with the port 104 to collect the fluid sample or to deliver therapeutics. In other embodiments, where the port 104 may be accessible at or above the surface 108 of the patient's 100 skin, the needle 112, a syringe, or other component may be used to access the port 104 via the septum 126. The needle 112 (or syringe) may couple to a reservoir 116 which may collect the fluid sample from the port 104 or deliver therapeutics to the port 104. In other embodiments, the reservoir 116 may be integrated or positioned near the pump 114. For example, the pump 114 may automatically obtain fluid samples periodically and store such fluid samples in the reservoir 116. In other examples, the pump 114 may automatically deliver therapeutics to the patient from the reservoir 116. It will be appreciated that in some embodiments that the fluid sampling system 102 may not include the reservoir 116. For example, in some embodiments, a syringe may be coupled to the port 104 (whether directly or via the needle 112) and may collect the fluid sample or deliver the therapeutics.

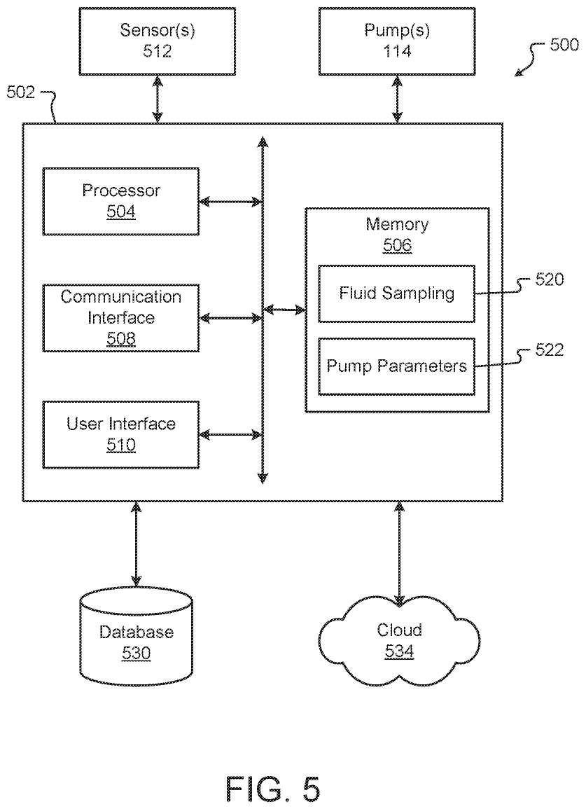

Turning to FIG. 5, a block diagram of a system 500 according to at least one embodiment of the present disclosure is shown. The system 500 may be used to sample a fluid from a patient such as the patient 100 and/or carry out one or more other aspects of one or more of the methods disclosed herein. The system 500 comprises a computing device 502, one or more sensors 512, one or more pumps 114, a database 530, and/or a cloud or other network 534. Systems according to other embodiments of the present disclosure may comprise more or fewer components than the system 500. For example, the system 500 may not include one or more components of the computing device 502, the sensor 512, the pump 114, the database 530, and/or the cloud 534.

The sensor(s) 512 may be used to track various parameters of a fluid sampling such as, for example, flow rate of the fluid sampling, a fluid volume, etc. The sensor(s) 512 may comprise a flow rate sensor (e.g., a flow meter), a pinwheel sensor, or any other sensor capable of measuring fluid volume and/or a flow rate. The sensor 512 may include one or more or any combination of components that are electrical, mechanical, electro-mechanical, magnetic, electromagnetic, or the like. In some embodiments, the sensor 512 may include a memory for storing sensor data. In still other examples, the sensor 512 may output signals (e.g., sensor data) to one or more sources (e.g., the computing device 502).

The sensor 512 may be positioned adjacent to or integrated with another component of the system 500 such as, but not limited to, the pump 114, the port 104, the catheter 106, and/or the computing device 502. In some embodiments, the sensor 124 is positioned as a standalone component. The sensor 512 may include a plurality of sensors and each sensor may be positioned at the same location or a different location as any other sensor. It will be appreciated that in some embodiments the sensor(s) 512 can be positioned at or on any component of the system 500 or environment (e.g., on any portion of the pump 114, the port 104, the catheter 106, and/or any other component at the fluid sampling system 102).

The computing device 502 comprises a processor 504, a memory 506, a communication interface 508, and a user interface 510. Computing devices according to other embodiments of the present disclosure may comprise more or fewer components than the computing device 502.

The processor 504 of the computing device 502 may be any processor described herein or any similar processor. The processor 504 may be configured to execute instructions stored in the memory 506, which instructions may cause the processor 504 to carry out one or more computing steps utilizing or based on data received from the sensor 512, the pump 114, the database 530, and/or the cloud 534.

The memory 506 may be or comprise RAM, DRAM, SDRAM, other solid-state memory, any memory described herein, or any other tangible, non-transitory memory for storing computer-readable data and/or instructions. The memory 506 may store information or data useful for completing, for example, any step of the method 600 described herein, or of any other methods. The memory 506 may store, for example, instructions and/or machine learning models that support one or more functions of the sensor 512 and/or the pump 114. For instance, the memory 506 may store content (e.g., instructions and/or machine learning models) that, when executed by the processor 504, enable fluid sampling 520. The fluid sampling 520 may enable the processor 504 to generate instructions for sampling a fluid from a patient based on, for example, input from the sensor 512, user input, information about a patient from which the fluid is sampled from, desired type of fluid sampled, an amount of fluid to sample, etc. The fluid sampling 520 may also enable the processor 504 to generate one or more pump parameters 522 for the pump 114. Such content, if provided as an instruction, may, in some embodiments, be organized into one or more applications, modules, packages, layers, or engines. Alternatively or additionally, the memory 506 may store other types of content or data (e.g., machine learning models, artificial neural networks, deep neural networks, etc.) that can be processed by the processor 504 to carry out the various method and features described herein. Thus, although various contents of memory 506 may be described as instructions, it should be appreciated that functionality described herein can be achieved through use of instructions, algorithms, and/or machine learning models. The data, algorithms, and/or instructions may cause the processor 504 to manipulate data stored in the memory 506 and/or received from or via the sensor 512, the pump 114, the database 530, and/or the cloud 534. The memory 506 may also be used to store, for example, the pump parameters 522 for operating the pump 114.

The computing device 502 may also comprise a communication interface 508. The communication interface 508 may be used for receiving image data or other information from an external source (such as the sensor 512, the pump 114, the database 530, the cloud 534, and/or any other system or component not part of the system 500), and/or for transmitting instructions, images, or other information to an external system or device (e.g., another computing device 502, sensor 512, the pump 114, the database 530, the cloud 534, and/or any other system or component not part of the system 500). The communication interface 508 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.55a/b/g/n, Bluetooth, NFC, ZigBee, and so forth). In some embodiments, the communication interface 508 may be useful for enabling the device 502 to communicate with one or more other processors 504 or computing devices 502, whether to reduce the time needed to accomplish a computing-intensive task or for any other reason.

The computing device 502 may also comprise one or more user interfaces 510. The user interface 510 may be or comprise a keyboard, mouse, trackball, monitor, television, screen, touchscreen, and/or any other device for receiving information from a user and/or for providing information to a user. The user interface 510 may be used, for example, to receive a user selection or other user input regarding any step of any method described herein. Notwithstanding the foregoing, any required input for any step of any method described herein may be generated automatically by the system 500 (e.g., by the processor 504 or another component of the system 500) or received by the system 500 from a source external to the system 500. In some embodiments, the user interface 510 may be useful to allow a medical professional or other user to modify instructions to be executed by the processor 504 according to one or more embodiments of the present disclosure, and/or to modify or adjust a setting of other information displayed on the user interface 510 or corresponding thereto.

Although the user interface 510 is shown as part of the computing device 502, in some embodiments, the computing device 502 may utilize a user interface 510 that is housed separately from one or more remaining components of the computing device 502. In some embodiments, the user interface 510 may be located proximate one or more other components of the computing device 502, while in other embodiments, the user interface 510 may be located remotely from one or more other components of the computer device 502.

The database 530 may store information about a patient, pump parameters 522, and/or information about desired fluid sample(s). The database 530 may be configured to provide any such information to the computing device 502 or to any other device of the system 500 or external to the system 500, whether directly or via the cloud 534.

The cloud 534 may be or represent the Internet or any other wide area network. The computing device 502 may be connected to the cloud 534 via the communication interface 508, using a wired connection, a wireless connection, or both. In some embodiments, the computing device 502 may communicate with the database 530 and/or an external device (e.g., a computing device) via the cloud 534.

The system 500 or similar systems may be used, for example, to carry out one or more aspects of the method 600 described herein. The system 500 or similar systems may also be used for other purposes.

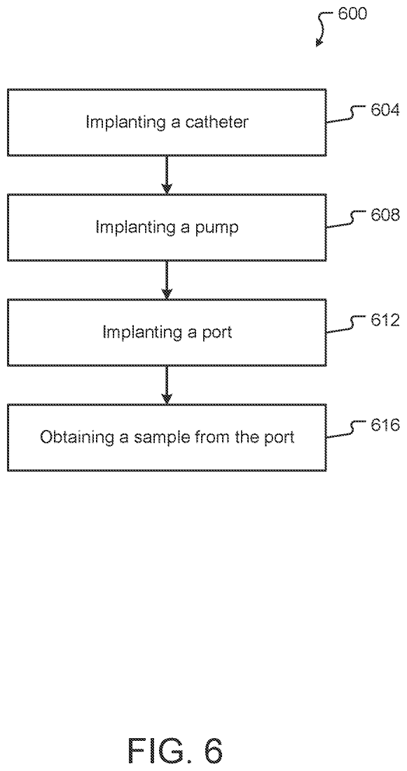

FIG. 6 depicts a method 600 that may be used, for example, sampling a fluid.

One or more steps of the method 600 (and/or one or more steps thereof) may be carried out or otherwise performed, for example, by at least one processor. One or more other steps of the method 600 may be carried out by a user such as a surgeon or other medical provider. The at least one processor may be the same as or similar to the processor(s) 504 of the computing device 502 described above. A processor other than any processor described herein may also be used to execute the method 600. The at least one processor may perform the method 600 by executing elements stored in a memory such as the memory 506. The elements stored in memory and executed by the processor may cause the processor to execute one or more steps of a function as shown in method 600. One or more portions of a method 600 may be performed by the processor executing any of the contents of memory, such as fluid sampling 520.

The method 600 comprises implanting a catheter (step 604). The catheter may be the same as or similar to the catheter 106 of a fluid sampling system such as the fluid sampling system 102. As previously described, the catheter may be implanted in a patient such as the patient 100 and may enable fluid sampling from the patient. The catheter may be implanted in a spinal region or a brain region of the patient. More specifically in some embodiments, the catheter may be implanted intrathecally.

The method 600 also comprises implanting a pump (step 608). The pump may be the same as or similar to the pump 114. The pump may be implanted, for example, anywhere on the patient. In some embodiments, the pump is implanted near the spine of a patient. In other embodiments, the pump is implanted near the abdomen of the patient and in still other embodiments, the pump may be implanted near or on the arm of a patient. The pump is configured to pump fluid sample to a port (described below) via the catheter.

It will be appreciated that in some embodiments the method may not include the step 608. The fluid sampling system may not include the pump, or the pump may be positioned external to the patient.

The method 600 also comprises implanting a port (step 612). The port may be the same as or similar to the port 104. The port may be implanted, for example, subcutaneously anywhere on the patient. In some embodiments, the port is implanted near the spine of a patient. In other embodiments, the port is implanted near the abdomen of the patient and in still other embodiments, the port may be implanted near or on the arm of a patient. The port is configured to be in fluid communication with the catheter and enables delivery of the fluid sample from the catheter.

The method 600 also comprises obtaining a fluid sample from the port (step 616). The fluid sample may be, for example, cerebrospinal fluid. The fluid sample may be obtained automatically using, for example, a computing device such as the computing device 502 or a controller capable of controlling, for example, the pump. A processor such as the processor 504 of the computing device may execute a fluid sampling such as the fluid sampling 520 to determine one or more parameters of the pump such as the pump parameters 522. The processor may receive data from one or more sensors such as the sensors 512 as input and may use the data to determine the pump parameters using the fluid sampling. The processor may also determine a desired sample fluid volume. Obtaining the fluid sample from the port may include causing the pump to pump the fluid sample from a desired region of the patient where the catheter is implanted to the port. In some embodiments, the pump may be automatically operated using the pump parameters. Obtaining the fluid sample from the port may also include using a needle such as the needle 112 to obtain the fluid sample from the port. More specifically, in embodiments where the port is implanted subcutaneously in the patient, the needle may be used to reach and penetrate a septum such as the septum 126 of the port through the patient's skin.

It will be appreciated that the step 616 may be repeated multiple times to obtain multiple fluid samples from the port.

The present disclosure encompasses embodiments of the method 600 that comprise more or fewer steps than those described above, and/or one or more steps that are different than the steps described above. For example, the method 600 may not include the step 608. The present disclosure also encompasses embodiments of the method 600 wherein the steps are performed in any order. For example, the step 612 may occur prior to the step 608.

As noted above, the present disclosure encompasses methods with fewer than all of the steps identified in FIG. 6 (and the corresponding description of the method 600), as well as methods that include additional steps beyond those identified in FIG. 6 (and the corresponding description of the method 600). The present disclosure also encompasses methods that comprise one or more steps from one method described herein, and one or more steps from another method described herein.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system for sampling fluid comprising:
a port implanted in a patient; and
a catheter implanted in the patient and in fluid communication with the port, the catheter enabling fluid sampling from the patient and delivery of the fluid sample via the port.

2. The system of claim 1, wherein the fluid is sampled from a patient having glioma.

3. The system of claim 1, wherein the fluid sampled is cerebrospinal fluid.

4. The system of claim 1, wherein the catheter is implanted to sample fluid from at least one of a spinal region or a brain region of the patient.

5. The system of claim 1, wherein the implanted port is implanted subcutaneously.

6. The system of claim 1, wherein the port comprises a septum in fluid communication with the catheter, and the system further comprises a needle configured to pierce the septum to collect the fluid sample.

7. The system of claim 1, further comprising a pump configured to pump the fluid sample to the port via the catheter.

8. The system of claim 7, wherein the pump is implanted in the patient.

9. A system for sampling cerebrospinal fluid comprising:
a port implanted in a patient;
a catheter implanted in the patient and in fluid communication with the port, the catheter enabling sampling cerebrospinal fluid from the patient and delivery of the cerebrospinal fluid via the port; and
a pump configured to pump the cerebrospinal fluid to the port via the catheter.

10. The system of claim 9, wherein the pump is implanted in the patient.

11. The system of claim 9, wherein the catheter is implanted to sample cerebrospinal fluid from at least one of a spinal region or a brain region of the patient.

12. The system of claim 9, further comprising a needle configured to selectively couple to the port to collect the fluid sample.

13. The system of claim 9, wherein the implanted port is implanted subcutaneously.

14. The system of claim 9, wherein the cerebral spinal fluid is sampled from a patient with glioma.

15. A method for sampling cerebral spinal fluid comprising:
implanting a catheter in a patient, the catheter in fluid communication with the cerebral spinal fluid to be sampled;
implanting a port subcutaneously, the port in fluid communication with the catheter; and
obtaining a sample of the cerebral spinal fluid from the port.

16. The method of claim 15, wherein obtaining the sample from the port comprises causing a pump to pump the cerebral spinal fluid from the spinal region to the port via the catheter.

17. The method of claim 16, wherein the pump is implanted in the patient.

18. The method of claim 16, wherein the sample of the cerebral spinal fluid is obtained from the port using a needle.

19. The method of claim 15, wherein the catheter is implanted in at least one of a spinal region or a brain region of the patient.

20. The method of claim 15, wherein the cerebral spinal fluid is sampled from a patient with glioma.
